(19) United States
(12) Patent Application Publication
(10) Pub. No.: US 2019/0091258 A1
(43) Pub. Date: Mar. 28, 2019

(54) THERAPEUTIC MIXTURES FOR TREATING OSTEOARTHRITIS COMPRISING NANO HEXAGONAL BORON NITRIDE COMPOSITION

(71) Applicant: BORTEK BOR TEKNOLOJILERI VE MEKATRONIK SANAYI TICARET ANONIM SIRKETI, Eskisehir (TR)

(72) Inventors: Suleyman Ay, Eskisehir (TR); Nuran Ay, Eskisehir (TR); Yapincak Goncu, Eskisehir (TR)

(21) Appl. No.: 16/081,466

(22) PCT Filed: Feb. 24, 2017
(86) PCT No.: PCT/TR2017/000033

§ 371 Date: Aug. 31, 2018

(30) Foreign Application Priority Data
Mar. 2, 2016 (TR) 2016/02710
May 12, 2016 (TR) 2016/06270

(51) Int. Cl. A61K 33/22; A61K 47/44; A61K 9/00; A61P 19/02

(52) U.S. Cl. CPC A61K 33/22; A61P 19/02; A61K 9/0014; A61K 47/44

(57) ABSTRACT

This invention relates to the mixtures employed in the treatment of the osteoarthritis, said mixtures being formed by the materials comprising the biocompatible solutions, lotions, creams, materials that contains chemical oils, materials that contains animal oils, materials that contains vegetable oils, ointments, gels, foams, plasters, compounds for enhancing the skin penetration and pomades, which are able to be applied topically to the skin, along with the therapeutic nano hexagonal boron nitride (hBN) composition.

This invention relates to the mixtures employed in the treatment of the osteoarthritis, said mixtures being formed by the materials comprising the biocompatible solutions, lotions, creams, materials that contains chemical oils, materials that contains animal oils, materials that contains vegetable oils, ointments, gels, foams, plasters, compounds for enhancing the skin penetration and pomades which are able to be applied topically to the skin, along with the therapeutic nano hexagonal boron nitride (hBN) composition. When said mixtures are applied to the skin, they penetrate the skin and reach the cartilage and bone ends. The mixture coats the cartilage and fills in the pores in the bone. The nano hBN is a biocompatible solid lubricant and prevents infection. The nano hBN composition lubricates the bone ends to facilitate bone movements, reduces the cartilage loss.

Osteoarthritis is a joint disease involving the destruction or loss of cartilage. The cartilage is a protein-containing material, which serves the function of a pad between the joints of the bones. The osteoarthritis is believed to result from the mechanical stress and the inflammatory processes. The development of the disease includes the loss of cartilage and the effect of this loss on the underlying bone. The extreme states of the disease enormously limit and obstruct a patient's life. The patients with osteoarthritis suffer from depression, incapability, limitations in performance and difficulty in performing the daily activities.

The pills, syrups, creams and lotions are used in the treatment of osteoarthritis. These include the following:
A- Analgesics: These are the analgesics that include acetaminophen, opioids (narcotics) and the atypical opioid called tramadol.
B- Nonsteroidal anti-inflammatory drugs: These are the most commonly employed drugs for relieving the inflammations and the pain associated with inflammation and include aspirin, ibuprofen, naproxen and celecoxib.
C- Corticosteroids: These are very powerful anti-inflammatory agents. These are administered orally in the form of pills or are directly injected into the joint.
D- Lubricant-Hyaluronic acid injection: Hyaluronic acid naturally forms in the joints and serves the function of a lubricant and shock absorber. However, hyaluronic acid is subject to breakdown in the patients with osteoarthritis. The injection of hyaluronic acid may serve the function of a pad in the knee to relieve the pain.

The patents related to the subject matter are also mentioned below:

The patent no. US20100016257 relates to the treatment of osteoarthritis by way of injecting the biodegradable and biocompatible microparticles made from natural and synthetic materials and polymers, which are carried in an aqueous vehicle, into the local area where the osteoarthritis is present.

The U.S. Pat. No. 8,765,714 relates to the oral or intra-articular formulations of sulphated hyaluronic acid, which are effective in the treatment of osteoarthritis and which do not cause inflammation.

The U.S. Pat. No. 8,779,006 describes that a pharmaceutical composition comprising sodium bicarbonate and calcium gluconate may be used for the prevention and treatment of osteoarthritis.

The U.S. Pat. No. 6,346,519 relates to the use of a composition comprising a nitric oxide synthase inhibitor and amino sugars in the treatment of osteoarthritis.

The patent no. CN104474110 describes the treatment of osteoarthritis with a Chinese medical mixture comprising mustard seeds, rehmannia root, millettia, cinnamon, cyathula, ephedra and sichuan.

The patents and applications mentioned above reduce the pain and inflammation temporarily or provide temporary lubrication in the joints for the treatment of osteoarthritis. The therapy is not long lasting or permanent.

The mixtures described in the present invention comprise a biocompatible solid lubricant, which simultaneously facilitates the bone movements by way of lubrication, is absorbed by the bone and cartilage, reduces the cartilage loss and prevents infection. The mixtures described in the present invention increase the lubrication while treating the inflammation and provide a long term cure for the region of osteoarthritis. The mixtures described in the present invention, owing to the presence of the solid lubricant, provide a therapy that is much more effective and permanent as compared to the other practices.

With this invention, the mixtures of materials comprising the biocompatible solutions, lotions, creams, ointments, gels, foams, plasters, pomades, compounds for enhancing the skin penetration and chemical, animal and vegetable oils, which are able to be applied topically to the skin, along with the solid lubricant, i.e. nano hexagonal boron nitride (hBN) composition, are used in the treatment of the osteoarthritis. When the mixtures are externally applied to the skin, the nano-size hBN composition penetrates the skin to fill in the smallest pores on the exposed bone and becomes located in the mass of the cartilage. Nano hBN composition is a biocompatible solid lubricant and anti-infective agent. Thus, it improves the lubricating property for the destroyed or lost cartilage and exposed bone. In addition, it features the therapeutic effect for the resulting joint inflammation (arthritis).

According to the present invention, the composition comprising nano hBN with a particle size D50 of less than 200 nanometers is prepared together with the biocompatible vegetable, animal and chemical oils and the compounds for enhancing the skin penetration. The nano hBN composition comprises more than 90% by weight hBN and less than 10% by weight boron oxide. The composition comprising the nano-size hBN is mixed with the biocompatible vegetable, animal and chemical oils and the compounds for enhancing the skin penetration by means of mechanical and/or ultrasonic methods and the mixture is exfoliated. The exfoliated mixture contains less than 5% by weight of the composition that comprises nano hBN.

The exfoliated mixture is used directly as a therapeutic for the treatment of osteoarthritis.

The exfoliated mixture is mixed with a biocompatible solution at a ratio smaller than 20% and is used as a therapeutic solution for treating osteoarthritis.

The exfoliated mixture is mixed with a biocompatible lotion at a ratio smaller than 20% and is used as a therapeutic lotion for treating osteoarthritis.

The exfoliated mixture is mixed with a biocompatible ointment at a ratio smaller than 50% and is used as a therapeutic ointment for treating osteoarthritis.

The exfoliated mixture is mixed with a biocompatible cream at a ratio smaller than 50% and is used as a therapeutic cream for treating osteoarthritis.

The exfoliated mixture is mixed with a biocompatible gel at a ratio smaller than 50% and is used as a therapeutic gel for treating osteoarthritis.

The exfoliated mixture is mixed with a biocompatible plaster at a ratio smaller than 50% and is used as a therapeutic plaster for treating osteoarthritis.

The exfoliated mixture is mixed with a biocompatible pomade at a ratio smaller than 50% and is used as a therapeutic pomade for treating osteoarthritis.

The exfoliated mixture is mixed with a biocompatible foam at a ratio smaller than 50% and is used as a therapeutic foam for treating osteoarthritis.

When the prepared mixtures are applied topically to the skin, they penetrate the skin and reach the joint cartilage region and the bone ends. The nano hBN in the mixtures coats the cartilage and fills in the pores in the bone. The nano hBN is a biocompatible solid lubricant and anti-infective agent. The nano hBN composition lubricates the bone ends to facilitate bone movements, reduces the cartilage loss.

With this invention, it was determined in the studies applied on volunteers that biocompatible solutions, lotions, materials that contains chemical oils, materials that contains animal oils, materials that contains vegetable oils, creams, ointments, gels, foams, plasters, compounds for enhancing the skin penetration, pomades that includes nano hexagonal boron nitride treat the osteoarthritis in the joints when applied topically to skin.

The examples and the results of the tests performed on the volunteers include, but are not limited to, the following:

The therapeutic solution for treating the osteoarthritis was applied to a 60 year old volunteer with difficulty in walking, and after the therapy for one month, the person's walking returned to normal and the person became free of the problem.

The therapeutic ointment for treating the osteoarthritis was applied for 15 days to a volunteer over the age of 50 with difficulty in going up the stairs, and the person became able to easily go up the stairs.

A 65 year old volunteer with difficulty in walking used the therapeutic cream for treating the osteoarthritis for 2 months and the person's walking returned to normal.

1. Therapeutic mixtures for treating the osteoarthritis developing in the knee, hip, shoulder, wrist, ankle, elbow, finger, toe, backbone and similar joints, said mixtures being formed by the materials comprising the biocompatible solutions, lotions, creams, materials that contains chemical oils, materials that contains animal oils, materials that contains vegetable oils, ointments, gels, foams, plasters, compounds for enhancing the skin penetration and pomades, which are able to be applied topically to the skin, along with the therapeutic nano hexagonal boron nitride (hBN) composition.

2. Therapeutic mixtures for treating the osteoarthritis according to claim 1 characterized in that the nano hBN-containing composition in the mixtures comprises more than 90% by weight nano hBN and less than 10% by weight boron oxide.

3. Therapeutic mixtures for treating the osteoarthritis according to claim 1 characterized in that the nano hBN used in the nano hBN composition in the mixtures has a particle size D50 of less than 200 nanometers.

4. Therapeutic mixtures for treating the osteoarthritis according to claim 1 characterized in that the nano-size hBN-containing composition in the mixtures is mixed with the biocompatible vegetable, animal and chemical oils and the compounds for enhancing the skin penetration by means of mechanical and/or ultrasonic methods and the mixture is exfoliated.

5. Exfoliated mixture according to claim 4 characterized in that it contains less than 5% by weight of the composition that comprises nano hBN.

6. Exfoliated mixture according to claim 4 characterized in that the exfoliated mixture is able to be used directly.

7. Exfoliated mixture according to claim 4 characterized in that the exfoliated mixture is mixed with a biocompatible solution at a ratio smaller than 20%.

8. Exfoliated mixture according to claim 4 characterized in that the exfoliated mixture is mixed with a biocompatible lotion at a ratio smaller than 20%.

9. Exfoliated mixture according to claim 4 characterized in that the exfoliated mixture is mixed with a biocompatible ointment at a ratio smaller than 50%.

10. Exfoliated mixture according to claim 4 characterized in that the exfoliated mixture is mixed with a biocompatible cream at a ratio smaller than 50%.

11. Exfoliated mixture according to claim 4 characterized in that the exfoliated mixture is mixed with a biocompatible gel at a ratio smaller than 50%.

12. Exfoliated mixture according to claim 4 characterized in that the exfoliated mixture is mixed with a biocompatible plaster at a ratio smaller than 50%.

13. Exfoliated mixture according to claim 4 characterized in that the exfoliated mixture is mixed with a biocompatible pomade at a ratio smaller than 50%.

14. Exfoliated mixture according to claim 4 characterized in that the exfoliated mixture is mixed with a biocompatible foam at a ratio smaller than 50%.
